(19) United States
(12) Patent Application Publication
(10) Pub. No.: US 2018/0182483 A1
(43) Pub. Date: Jun. 28, 2018

(54) LOGISTICS SYSTEM AND PROCESS FOR STREAMLINING SURGERIES WITHIN A HOSPITAL AND OTHER WORKFLOW ENVIRONMENTS THAT OPERATE IN A VENDOR/MANUFACTURER MODEL

(71) Applicant: Joseph Testa, Wellington, FL (US)

(72) Inventor: Joseph Testa, Wellington, FL (US)

(21) Appl. No.: 15/850,946

(22) Filed: Dec. 21, 2017

Related U.S. Application Data
(60) Provisional application No. 62/438,581, filed on Dec. 23, 2016.

(51) Int. Cl. G16H 40/40; G16H 40/20; G06Q 10/06

(52) U.S. Cl. CPC G16H 40/40; G16H 40/60; G06Q 10/0631; G16H 40/20

(57) ABSTRACT

A logistics system and process for implementation within a workflow environment that operates under a vendor/manufacturer model. A user friendly app for a smart phone, electronic tablet or other computer device is utilized by employees, approved vendors and professionals/management and other facilitators in the workflow environment in conjunction with RFID technology, a color coded timeline, a locator and tracking system, an inventory storage, billing and reordering system and a color coded metric system. The logistics system and process enables the vendor to save money by eliminating or reducing high salary sales representatives.

This non-provisional patent application is based on provisional patent application Ser. No. 62/438,581 filed Dec. 23, 2016.

### BACKGROUND OF THE INVENTION

### Field of the Invention

The present invention relates to a logistics system and process for improving efficiency and reducing costs in a workflow environments and, more particularly, to a logistics system and process that is particularly suited for use in a workflow environment that operates under a vendor/manufacturer model wherein professionals/management and employees in the workflow environment need to track and use inventory of certain products, equipment, devices and supplies that are purchased from approved vendors/manufacturers on a regular basis.

### Discussion of the Related Art

In a workflow environment, such as a hospital setting, there is a continual need for various products, equipment, devices and other supplies from vendors/manufacturers. For instance, in a hospital setting, the various departments in the hospital must communicate with one another throughout the process of emitting a patient for emergency room/operating room surgery right through the discharge of the patient from the hospital. Throughout this process, it may be necessary to obtain certain products, devices or other items from a vendor/manufacturer, such as an implant device, as well as surgery instruments and other products from the vendor/manufacturer. To accomplish this, a sales representative usually acts as a middleperson or liaison between the vendor/manufacturer and the hospital. The sales representative communicates closely with the physicians, management and other staff members of the hospital in the process of the hospital purchasing the necessary devices, supplies and other equipment from the vendor/manufacturer. Naturally, the sales representative is working for the vendor/manufacturer in order to sell the hospital the appropriate devices, supplies, equipment, and the like. With each sale of such items, the vendor/manufacturer must pay the sales representative a commission and/or a fixed salary. The use of sales representatives in this type of setting, as well as other workflow environments, can be very costly to the vendor/manufacturer. Moreover, use of sales representatives in a workflow environment can slow down the process and limit the business of the workflow environment to fewer choices of vendors/manufacturers as well as products.

Accordingly, there is a need for a logistics system and process that enables a vendor/manufacturer to save money on paying very high salaries and commissions to sales representatives, while also allowing the business of the workflow environment to reduce their costs and to streamline the logistical flow of operations/tasks in the workflow environment.

### SUMMARY OF THE INVENTION

This present invention is directed to a logistics system and process for implementation within a workflow environment that operates under a vendor/manufacturer model. A user friendly app for a smart phone, electronic tablet or other computer device is utilized by employees, approved vendors and professionals/management and other facilitators in the workflow environment in conjunction with RFID technology, a color coded timeline, a locator and tracking system, an inventory storage, billing and reordering system and a color coded metric system. The logistics system and process enables the vendor to save money by eliminating or reducing high salary sales representatives.

### BRIEF DESCRIPTION OF THE DRAWINGS

For a fuller understanding of the nature of the present invention, reference should be made to the following detailed description taken in conjunction with the accompanying drawings in which:

FIGS. 1-3 are schematic diagrams illustrating the flow of operations through the logistical system and process of the present invention in a hospital environment using the specific example of a patient entering the emergency room of the hospital with a fractured hip, wherein the patient requires hip replacement surgery.

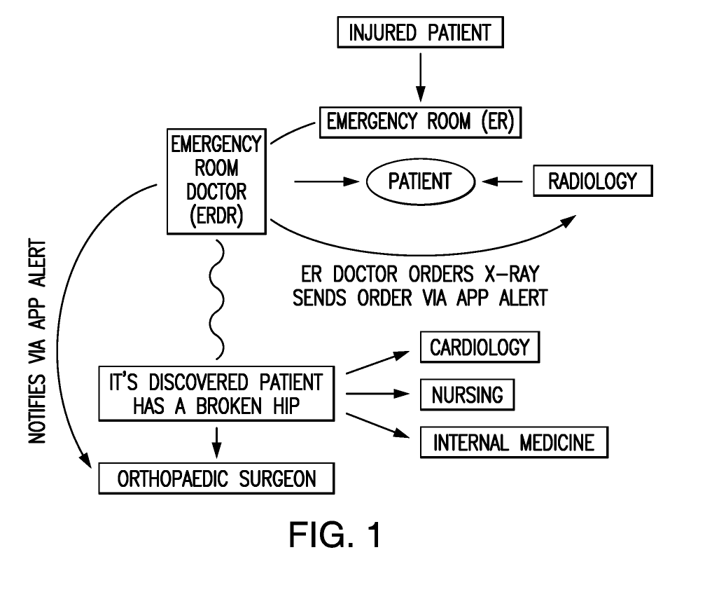

### DETAILED DESCRIPTION OF THE PREFERRED EMBODIMENT

A logistics system which implements a process to help streamline surgeries within a hospital system.

This system is not limited to Orthopaedics but can span across all workflow systems that operate in a vendor/manufacturer model. An Orthopaedic/Hospital model is simply used herein as an example of an application of the logistics system and process of the present invention which can logistically solve both a management and cost model from the manufacturer/distributor end and the customer/consumer end.

The example of the logistics system in an orthopaedic/Hospital model includes the following features:
operating system
APP for smart phone, tablet (iPad), computer
RFID technology
color coded timeline
tray locator
patient logistics system
color coded metric system for Operating Room (OR) flow
PYXIS™ system model/smart implant storage
reordering system
billing
patient tracking
X-ray templating/access/ct/MRI

The idea is to take a patient all the way through the hospital process from entry/admission into the Emergency Room (ER)/Operating Room (OR) (trauma/elective cases) to exit of the hospital (discharge).

This includes, but is not limited to, instruments and plants needed to be booked for the surgeries.

A user friendly smart application can be utilized by the hospital Staff/Approved Vendors/Physicians with hospital privileges.

Example: patient enters the hospital through the ED (Emergency Department). Patient has fallen down and potentially broken their hip. Emergency Department physician puts in orders through the application for patient to be sent to radiology to have X-rays taken.

At this point the orthopedic physician on call is notified and can go into the application either by phone/tablet/computer to view patient's current status, location, and charts/xrays/CT SCAN/MRI.

It is identified that the patient has a broken hip (Subtroch fracture).

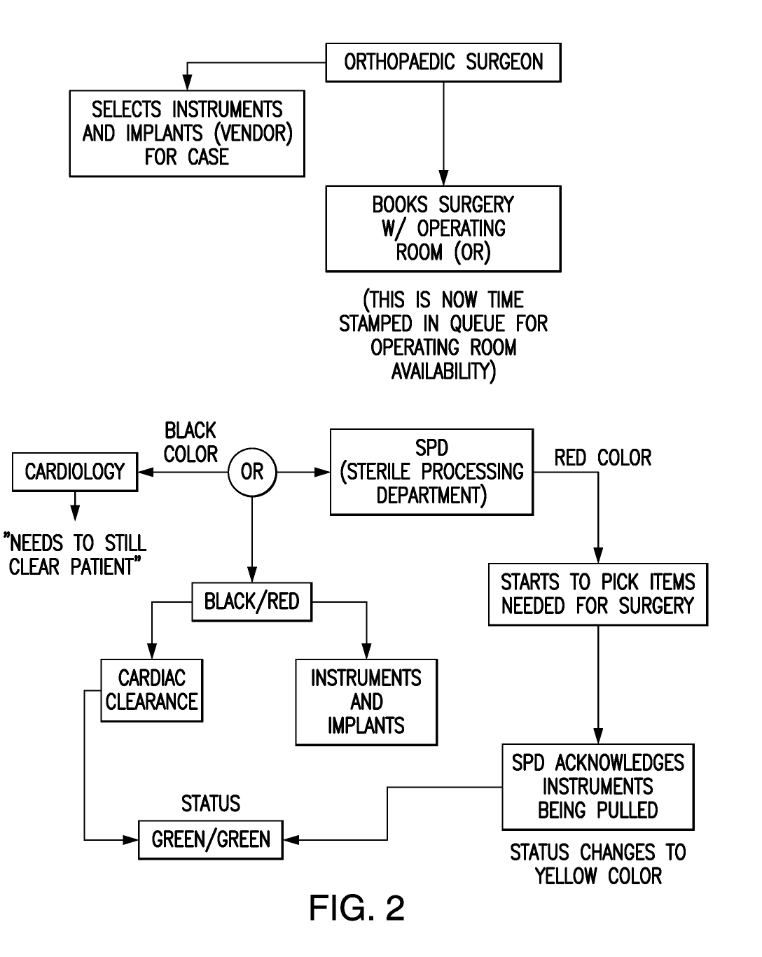

Referring to FIG. 2, the Orthopaedic surgeon can now select the instruments and implants for the case which he/she wants to have cleared for surgery at 4:00 pm. The case is then sent to a que in the Operating Room/Sterile Processing Department (OR/SPD), TV screen/monitor.

This will trigger a color coded process that shows what point the case is in the process of being picked.

This triggers a BLACK COLOR because a cardiologist needs to see the patient since the patient hasn't had any cardiac clearance.

Since the case was just added the color is RED which indicates a new case has been added on and nothing has been done.

The patient now has two colors. Black/Red.

In order for the surgery to move forward the patient needs a Green/Green status.

Immediately, the SPD staff knows to pull the instruments needed to provide the surgeon for the upcoming surgery. A preference card is generated from the system to help assist in this process.

Cardiology clears and status changes from black to green.

Case cart is now being pulled by staff in SPD. Status changes Red to Yellow.

Once everything is on the cart which is required for the case the staff member changes the status from Yellow to Green.

We now have an indication of Green/Green status. This means the OR is prepared for surgery once a room and team is available.

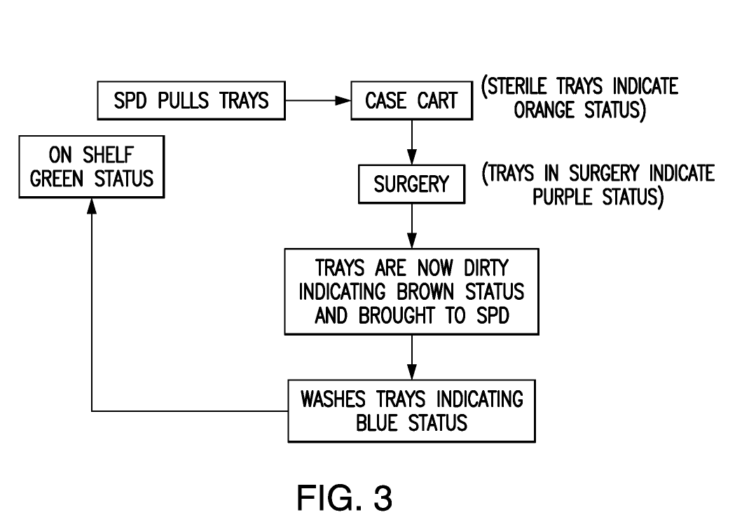

Referring to FIG. 3, the vendor and hospital owned trays are monitored using RFID or similar technology built into the trays.

This tracks the progress/status of where the tray is located and if the tray is in a case/sterile/being washed/dirty.

Also a color code system.
In case—purple
Sterile—orange
Being washed—blue
Dirty—brown

Implants can be kept in a PYXIS™ or similar System

Vendors will no longer be as crucial to the OR process because the majority of the trauma and elective cases can be run by the hospital team.

Many of the daily duties of the vendors can be eliminated using the logistics system and process of the present invention.

Implants are tracked and monitored through PYXIS™ system to ensure proper sizing, expiration date, and anatomic specifications.

SPD/Nurses/OR Staff are all trained by vendor to put trays together after each case to ensure trays are properly turned over.

Vendors will send implants directly to the hospital via FedEx/UPS or courier using a “Just-in-time” method of delivery.

Train OR staff to utilize this application to enter data both on equipment usage and billable charges.

(Example: electronic surgery sheet tracks patient information and manages the accounts billing process for payment tracking and reordering.) Data is entered into the application to reorder implants and disposables to be shipped next day to the hospital.

OR staff will be trained to clean and reorganize instruments/trays in a logistical manner to effectively and efficiently reprocess the instruments needed in order to reuse them for another similar surgery.

OR staff will be trained to locate and replenish the inventory for implants used during the surgeries from the following day.

The logistics system and process of the present invention enables the vendor to save money on paying very high salaries to sales representatives, while also allowing hospitals to reduce their implant costs and to streamline OR logistical flow.

What is claimed is:

1. A process for improving efficiency in a workflow environment comprising the steps of:
providing computer application software for use on at least one computer device;
establishing approved vendors and manufacturers of products including devices, instruments, equipment and supplies;
moving a subject through the workflow environment;
tracking the subject through different stages of the workflow environment using the computer application software running on the at least one computer device to determine the current status and location of the subject, as well as data relating to the subject;
determining tasks, operations or procedures to be performed relating to the subject;
selecting specific products such as devices, instruments, equipment and supplies that are needed for performing the tasks, operations or procedures and entering the selected products in the computer application software using the at least one computer device;
ordering the selected products for delivery to a specific location in the workflow environment;
assigning color codes for indicating the current status of the subject and the ordered products;
displaying the color codes on the at least one computer device running the computer application software;
using the color codes displayed on the at least one computer device for determining the readiness of the subject and the ordered products for beginning the tasks, operations or procedures to be performed;
tracking inventory of the products using the computer application software; and
reordering new products from the approved vendors.

2. The process as recited in claim 1 wherein the at least one computer device is a portable electronic computer device.

3. The process as recited in claim 2 further comprising:
a plurality of the portable electronic computer devices all operating on a computer network for allowing communication between the plurality of portable electronic computer devices.

4. The process as recited in claim 1 wherein the subject is a human.

5. The process as recited in claim 1 wherein the subject is an animal.

6. The process as recited in claim 1 wherein the subject is an object.

7. The process as recited in claim 1 wherein the workflow environment is in a hospital.

8. The process as recited in claim 1 further comprising the step of:
tracking the location of the products in inventory and throughout the workflow environment using RFID technology.

9. The process as recited in claim 1 further comprising the steps of:
automatically entering billing amounts for each product ordered and for each task, operation or procedure performed.
